(19) United States
(12) Patent Application Publication
(10) Pub. No.: US 2013/0209379 A1
(43) Pub. Date: Aug. 15, 2013

(54) LIP COSMETIC FORMULATIONS

(71) Applicants: Matthew Mills, West Hills, CA (US); Allan Kurtz, Encino, CA (US)

(72) Inventors: Matthew Mills, West Hills, CA (US); Allan Kurtz, Encino, CA (US)

(73) Assignee: Pacific Shore Holdings, Inc, Chatsworth, CA (US)

(21) Appl. No.: 13/700,894

(22) PCT Filed: Jun. 16, 2011
(86) PCT No.: PCT/US11/40603

§ 371 Date: Feb. 5, 2013

Related U.S. Application Data
(60) Provisional application No. 61/355,509, filed on Jun. 16, 2010.

(51) Int. Cl. A61K 9/00; A61K 31/522; A61K 31/28; A61K 31/704

(52) U.S. Cl. CPC A61K 9/0053; A61K 31/704; A61K 31/522; A61K 31/28
USPC 424/59; 514/26; 424/64

(57) ABSTRACT

Cosmetic formulations suitable for application to the lips are described herein. The lip cosmetic formulations can contain at least caffeine, extract of Hoodia gordinii and extract of green tea. The lip cosmetic formulations can also contain a bioavailable chromium source and/or L-carnitine. Optionally, the lip cosmetic formulations can also contain a sunscreen.

### CROSS-REFERENCE TO RELATED APPLICATIONS

This application claims the benefit of priority under 35 U.S.C. §119 from U.S. Provisional Patent Application Ser. No. 61/355,509, filed Jun. 16, 2010, which is incorporated herein by reference in its entirety.

### STATEMENT REGARDING FEDERALLY SPONSORED RESEARCH OR DEVELOPMENT

Not applicable.

### BACKGROUND

The present disclosure generally relates to topical cosmetic formulations, and, more specifically, to cosmetic formulations that are suitable for application to the lips.

Cosmetic formulations that are suitable for application to the lips can include, for example, lipsticks, lip glosses and lip balms. In many cases, such lip cosmetic formulations can be used for appearance purposes only. In such instances, lip cosmetic formulations can contain ingredients that enhance the lip's physical characteristics (e.g., colorants). In addition to physical appearance, lip cosmetic formulations can serve in a protective or therapeutic role for the wearer's lips, in lieu of or in addition to the previously noted appearance characteristics. For example, lip cosmetic formulations can be used to protect the lips from sunlight, wind and cold. In this regard, lip cosmetic formulations can contain a variety of lipophilic materials such as, for example, fats, oils, waxes and emollients which soften the lips and protect against the effects of wind. In addition, lip cosmetic formulations can contain sunscreens that protect against the damaging effects of the sun. To enhance the comfort of the wearer, lip cosmetic formulations can contain still other ingredients such as, for example, flavorants and fragrances.

Although lip cosmetic formulations have been used in a protective or therapeutic role for a wearer's lips, it has been uncommon in the art to use lip cosmetic formulations as a delivery vehicle for more generalized therapy throughout the wearer's body. The lips are located adjacent to the buccal cavity of the mouth, which can adsorb a variety of beneficial in vivo therapeutic agents through the buccal and sublingual mucosa. Adsorption of therapeutic agents within the buccal cavity can be particularly advantageous compared to oral and gastric routes, since first-pass hepatic metabolism is avoided during buccal adsorption to give high circulating plasma levels of therapeutic agents. For lip cosmetic formulations, ingestion and adsorption within the buccal cavity can occur as a wearer routinely licks or bites his or her lips during the course of the day or during the consumption of food or beverages.

A number of natural dietary supplements are purportedly able to suppress a user's appetite and manage weight loss. Many of these natural dietary supplements are Generally Recognized as Safe (GRAS) as designated by Title 21 of the U.S. Code of Federal Regulations section 182. Such appetite suppressants can be taken one or more times daily, often at mealtimes. In order to achieve the most beneficial effects of the appetite suppressant, a user must remember to take the agent on a regular dosing schedule. Hunger can sometimes return between meals as circulating plasma levels of the appetite suppressant fall between doses, particularly if a regular dose is missed. Although a number of substances are known for their ability to suppress a user's appetite, most appetite suppressants fail to actively stimulate a user's metabolism to more effectively burn calories and fat to result in more effective weight loss.

In view of the foregoing, dietary supplements that supply appetite suppression and can actively stimulate weight loss would be of substantial benefit in the art. Dietary supplements that can provide these properties more continually than do conventional orally ingested supplements taken on a regular dosing schedule would be particularly beneficial in this regard. The present invention satisfies these needs and provides related advantages as well.

### SUMMARY

The present disclosure generally relates to topical cosmetic formulations, and, more specifically, to cosmetic formulations that are suitable for application to the lips.

In some embodiments, lip cosmetic formulations described herein include caffeine, extract of Hoodia gordinii and extract of green tea.

In some embodiments, lip cosmetic formulations described herein include caffeine, extract of Hoodia gordinii and extract of green tea, where a concentration of the caffeine, the extract of Hoodia gordinii and the extract of green tea each range between about 1.5% and about 2.0% by weight of the lip cosmetic formulation.

In some embodiments, lip cosmetic formulations described herein include caffeine, extract of Hoodia gordinii, extract of green tea and chromium picolinate, where a concentration of the caffeine, the extract of Hoodia gordinii and the extract of green tea each range between about 1.5% and about 2.0% by weight of the lip cosmetic formulation and a concentration of the chromium picolinate ranges between about 0.05% and about 0.5% by weight of the lip cosmetic formulation.

The foregoing has outlined rather broadly the features of the present disclosure in order that the detailed description that follows can be better understood. Additional features and advantages of the disclosure will be described hereinafter, which form the subject of the claims.

### DETAILED DESCRIPTION

The present disclosure generally relates to topical cosmetic formulations, and, more specifically, to cosmetic formulations that are suitable for application to the lips.

Obesity is the leading cause of lifestyle-related disease and death in the United States after smoking Between 1980 and 2000, the number of obese adult Americans increased by more than 50 percent according to a 2000 Center for Disease Control (CDC) study. Childhood and adolescent obesity has also become increasingly problematic in recent years, with the number of obese children and adolescents tripling between 1980 and 2000.

In various embodiments, the present disclosure is directed to lip cosmetic formulations that can influence a user's metabolism and/or appetite. By managing a user's metabolism and/or appetite, weight loss and weight management can be realized. In various embodiments, the lip cosmetic formulations described herein can contain at least caffeine, extract of Hoodia gordinii and extract of green tea. These active ingredients are all naturally occurring substances and can be classified as GRAS.

The lip cosmetic formulations of the present disclosure can allow the active ingredients contained therein to be adsorbed through the buccal cavity as a user licks or bites his or her lips or consumes food or beverages during the course of a day. In this regard, the present formulations can be particularly advantageous for appetite suppression and weight management. First, the active ingredients of the present formulations can be more continuously administered throughout the day without a conscious effort on the part of the user, as the licking and biting of one's lips often can occur without conscious thought, much like blinking one's eyelids. Second, because the active ingredients of the present formulations can be adsorbed through the buccal cavity, they are not subject to first-pass hepatic metabolism and/or other gastric degradation and, accordingly, can achieve higher circulating plasma levels than if a comparable dose was taken orally. In addition, peak plasma levels can be achieved much more rapidly with buccal cavity administration compared to oral administration, which can advantageously result in more rapid appetite suppression effects being realized. Buccal cavity administration can also avoid local toxic effects, such as those observed during administration through other mucosal surfaces such as, for example, the nasal mucosa. It is to be recognized, however, that some oral ingestion and adsorption can also occur due to swallowing that occurs during the time that buccal cavity adsorption takes place.

In addition to the foregoing advantages, the lip cosmetic formulations of the present disclosure can contain amounts of their active ingredients such that the formulations can be applied any number of times throughout the day, much more often than if a dietary supplement was only taken at mealtimes on a regular dosing schedule. In various embodiments, the lip cosmetic formulations described herein are suitable for being applied to the lips about 3 times a day, or about 5 times a day, or about 7 times a day, or about 9 times a day, or about 11 times a day, or about 13 times a day, or about 15 times a day, or more than about 15 times a day. The lip cosmetic formulations can be applied by a user in response to hunger or to prevent hunger. As many users routinely apply lip cosmetic formulations throughout the course of a normal day, the present formulations can result in increased patient compliance over that of other in vivo administration routes including oral administration, intravenous administration, intraperitoneal administration, intranasal administration or rectal administration.

In addition to the lips' location adjacent to the buccal cavity, their proximity to the nasal passages are also particularly advantageous in the present embodiments in which an appetite suppressant and/or weight loss supplement is/are supplied in a lip cosmetic formulation. As one of ordinary skill in the art will appreciate, the olfactory senses can regulate one's enjoyment of food and stimulate one's appetite. In this regard, the present lip cosmetic formulations can also contain agents that affect a user's olfactory enjoyment of food in order to suppress one's appetite and control weight. Appetite suppression through the olfactory system can be stimulated by a number of agents including, for example, flavoring agents and odorants that can influence the appetite centers of the brain and signal appetite satiation, for example.

As used herein, the term “lip cosmetic formulation” refers to a topical substance or combination of substances that is suitable for being applied to the lips. According to the various embodiments described herein, lip cosmetic formulations can result in a change in the physical appearance of a user's lips, although they are not required to do so. According to the various embodiments described herein, illustrative but non-limiting examples of lip cosmetic formulations can include, for example, lipsticks, lip glosses and lip balms.

As used herein, the term “buccal cavity” refers to the cavity defining the mouth, including the cheeks, lips and tongue, particularly the buccal mucosa and sublingual mucosa.

As used herein, the term “emollients” refers to substances that soften and smooth the lips, and the term “moisturizers” refers to substances that provide hydration to the lips. In the description that follows, the more general term “emollients” will be used unless otherwise noted. It is to be recognized that use of the term emollients will refer equivalently to both emollients and moisturizers. Further, it is to be recognized that emollients and moisturizers can be used in combination with one another in some embodiments.

In various embodiments, lip cosmetic formulations of the present disclosure can include a stimulant, an appetite suppressant and an activator of phosphodiesterase. The various components of the lip cosmetic formulations described herein can be obtained from natural sources or produced synthetically. When obtained from natural sources, other inactive substances or less active substances can also be present with the active substance (e.g., in the raw extract of a natural product). Although purported active substances are postulated in the disclosure that follows, it should be recognized that other substances from a natural source can be wholly or partially responsible for an extract's bioactivity. Still further, the disclosure of a particular mechanism of action for a given substance should not be considered limiting, and other mechanisms of bioactivity can be possible for a given compound, any of which can be beneficial for the present embodiments.

In various embodiments, lip cosmetic formulations of the present disclosure can contain caffeine, extract of Hoodia gordonii and extract of green tea. In some embodiments, the lip cosmetic formulations can further include a bioavailable chromium source such as, for example, chromium picolinate. In still further embodiments, the lip cosmetic formulations can contain L-carnitine. In some embodiments, a concentration of the caffeine, the extract of Hoodia gordonii and the extract of green tea can each range between about 0.5% and about 3.0% by weight of the lip cosmetic formulation.

In various embodiments, the lip cosmetic formulations described herein can include a stimulant such as, for example, caffeine. Although consumption of large quantities of caffeine can increase cortisol and insulin levels, both of which can result in weight gain instead of weight loss, consumption of more moderate quantities can promote weight loss, particularly when ingested prior to exercise. Without being bound by theory or mechanism, it is believed that the present formulations can benefit from the presence of caffeine due to its stimulant properties. The advantages of caffeine in this regard can be particularly profound when the present formulations are used in conjunction with exercise.

The lip cosmetic formulations of the present disclosure can contain caffeine in quantities that can be suitable for achieving the foregoing benefits of this substance. In some embodiments, the present formulations can contain between about 0.05% and about 3.0% caffeine by weight of the formulation, including all values and subranges therebetween. In some embodiments, the present formulations can contain between about 0.5% and about 3.0% caffeine by weight of the formulation. In some embodiments, the present formulations can contain between about 0.05% and about 1.5% caffeine by weight of the formulation. In other embodiments, the present formulations can contain between about 1.5% and about 3.0% caffeine by weight of the formulation. In still other embodiments, the present formulations can contain between about 1.5% and about 2.0% caffeine by weight of the formulation. In some embodiments, the present formulations can contain about 2.0% caffeine by weight of the formulation, or about 1.5% caffeine by weight of the formulation, or about 1.25% caffeine by weight of the formulation, or about 0.75% caffeine by weight of the formulation.

In various embodiments, the lip cosmetic formulations described herein can include an appetite suppressant such as, for example, extract of Hoodia gordonii. Hoodia gordonii has been traditionally used by the bushmen of South Africa for its appetite suppression properties. The question of whether Hoodia gordonii extracts actually have appetite suppression properties has been the subject of vigorous scientific debate. However, these questions have primarily been directed toward oral administration routes, which can be accompanied by a low bioavailability, rapid excretion and potential first-pass hepatic metabolism, none of which are considered to be problematic when adsorbed through the buccal cavity as in the present embodiments. Although there has been some disagreement as to the hepatic stability of Hoodia gordonii extracts, a recent study has demonstrated metabolic stability and intestinal transport of its primary active substance in an in vitro study.

Without being bound by theory or mechanism, it is believed that the primary active substance of Hoodia gordonii extract is a compound now commonly referred to in the art as P57, the structure of which is shown in Formula (1) below.

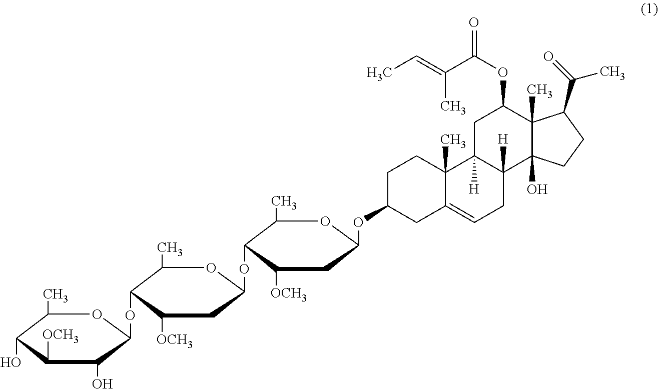

P57 comprises an oxypregnone steroidal glycoside, which is believed to be responsible for the appetite suppression properties of Hoodia gordonii extracts (see van Heerden, et al., Phytochemistry, 68:2007, pp. 2545-2453; van Heerden, et al., Journal of Ethnopharmacology, 119:2008, pp. 134-137; and Avula, et al., Journal of AOAC International, 89:2006, pp. 606-611). Again without being bound by theory or mechanism, it is believed that P57 can interact with the brain's hypothalamus, which can help regulate appetite (see MacLean, et al., Brain Research, 1020:2004, pp. 1-11). According to these studies, P57 can increase the ATP content of hypothalamic neurons by about 50%-150%. Further, these studies have shown that third ventricle administration of P57 can reduce subsequent 24-hour food intake by about 40%-60%. It is to be noted that ventricular administration of P57 avoids potential first-pass hepatic metabolism and bioavailability problems in a similar manner to that of the present embodiments.

In some embodiments, the present lip cosmetic formulations can contain isolated or enriched P57. When isolated or enriched P57 is used, the P57 can be isolated from natural Hoodia gordinii or produced synthetically by techniques known to one having ordinary skill in the art. In more preferred embodiments, the lip cosmetic formulations can contain natural extracts of Hoodia gordonii. That is, the active substances(s) of Hoodia gordonii can be present with one or more inactive or less active substances found in the natural material.

The lip cosmetic formulations of the present disclosure can contain extract of Hoodia gordonii in quantities that can be suitable for achieving the foregoing benefits of this substance. That is, the quantity of Hoodia gordonii extract can be sufficient to achieve appetite suppression in some embodiments. In some embodiments, the present formulations can contain between about 0.05% and about 3.0% extract of Hoodia gordonii by weight of the formulation, including all values and subranges therebetween. In some embodiments, the present formulations can contain between about 0.5% and about 3.0% extract of Hoodia gordonii by weight of the formulation. In some embodiments, the present formulations can contain between about 0.05% and about 1.5% extract of Hoodia gordonii by weight of the formulation. In other embodiments, the present formulations can contain between about 1.5% and about 3.0% extract of Hoodia gordonii by weight of the formulation. In still other embodiments, the present formulations can contain between about 1.5% and about 2.0% extract of Hoodia gordonii by weight of the formulation. In some embodiments, the present formulations can contain about 2.0% extract of Hoodia gordonii by weight of the formulation, or about 1.5% extract of Hoodia gordonii by weight of the formulation, or about 1.25% extract of Hoodia gordonii by weight of the formulation, or about 0.75% extract of Hoodia gordonii by weight of the formulation.

In some embodiments, the lip cosmetic formulations of the present disclosure can contain an activator of phosphodiesterase such as, for example, extracts of green tea. Green tea and its derivatives have long been considered a natural material that can assist in maintaining health. As used herein, green tea extract is a herbal derivative of green tea leaves (Camellia sinensis), which contains a number of antioxidant compounds, mainly catechins, also known as green tea polyphenols. Illustrative but non-limiting examples of green tea polyphenols can include, for example, epigallocatechin gallate (EGCG), epigallocatechin, epicatechin gallate and epicatechin Of these, EGCG is the generally the primary constituent of a green tea extract. Other constituents of green tea extract can include, but are not limited to, caffeine and flavonoids such as, for example, kaempferol, quercetin and myricetin. It should be noted that the caffeine in the present lip cosmetic formulations can come from both green tea extract and other caffeine sources. However, it is to be recognized that the total caffeine content of the present formulations is maintained within the ranges set forth above.

Without being bound by theory or mechanism, it is believed that the catechins of green tea extract can be responsible for its activation of phosphodiesterase. It should be noted that the activation can be suppressed by the presence of epinephrine, which suggests a weak binding of the β-adrenoreceptor (see Kuppusamy, et al., Biochem. Pharmacol., 44:1992, pp. 1307-1315). Further, one-month metabolism studies in mice have suggested that green tea catechins can increase acyl CoA oxidase quantities, medium chain acyl CoA dehydrogenase quantities, and β-oxidation liver activity, all of which can result in increased lipid catabolism (see Murase, et al., Int. J. Obes. Relat. Metab. Disord. 26:2002, pp. 1459-1464).

Diseases associated with elevated blood-lipid levels can include, for example, fatty liver disease, which can be caused by excessive intake of fat-containing foods and alcohol. Fatty liver disease can cause large amounts of lipids to be deposited in the liver tissue and can result in increased levels of serum glutamate-oxaloacetate transaminase (GOT), glutamate-pyruvate transaminase (GPT), and gamma-glutamyl transpeptidase (GTP) (see Banciu, et al., Med. Intern., 20:1982, pp. 69-71 and Par, et al., Acta. Med. Acad. Sci. Hung. 33:1976, pp. 309-319). In the process of developing a fatty liver, fat can accumulate in the liver mainly in the form of triglycerides and fatty acids and, to a minor extent, in the form of cholesterol. One of the major signs of fatty liver disease can be high blood cholesterol and/or triglyceride levels. In addition to the beneficial properties of green tea extract set forth above, it has also been reported that green tea extract can help alleviate elevated serum GOT and GPT levels, which can be beneficial for combating fatty liver disease and assisting in weight management (see Hayashi, et al., Nippon Yakuri Gaku Zasshi, 100:1992, pp. 391-399).

In some embodiments, the present formulations can contain a single or multiple isolated or enriched catechin(s). The isolated/enriched catechin(s) can be obtained from natural green tea or produced synthetically by techniques known to one having ordinary skill in the art. In more preferred embodiments, the present lip cosmetic formulations can contain natural extract of green tea. That is, the active substances(s) of green tea extract can be present with one or more inactive or less active substances found in the natural material.

The lip cosmetic formulations of the present disclosure can contain extract of green tea in quantities that can be suitable for achieving the foregoing benefits of this substance. That is, the quantity of green tea extract can be sufficient to improve phosphodiesterase activity in an embodiment or to reduce serum cholesterol and/or triglyceride levels in another embodiment. In some embodiments, the present formulations can contain between about 0.05% and about 3.0% extract of green tea by weight of the formulation, including all values and subranges therebetween. In some embodiments, the present formulations can contain between about 0.5% and about 3.0% extract of green tea by weight of the formulation. In some embodiments, the present formulations can contain between about 0.05% and about 1.5% extract of green tea by weight of the formulation. In other embodiments, the present formulations can contain between about 1.5% and about 3.0% extract of green tea by weight of the formulation. In still other embodiments, the present formulations can contain between about 1.5% and about 2.0% extract of green tea by weight of the formulation. In some embodiments, the present formulations can contain about 2.0% extract of green tea by weight of the formulation, or about 1.5% extract of green tea by weight of the formulation, or about 1.25% extract of green tea by weight of the formulation, or about 0.75% extract of green tea by weight of the formulation.

In some embodiments, the present lip cosmetic formulations can further include a bioavailable chromium source. Illustrative bioavailable chromium sources can include, for example, chromium chloride, chromium nicotinate and chromium picolinate. The picolinate ligand particularly improves the bioavailability of this trace nutrient, making it an especially advantageous bioavailable chromium source. However, one having ordinary skill in the art will appreciate that other chromium salts and chromium coordination compounds can be used equivalently to provide bioavailable chromium.

Bioavailable chromium sources, particularly chromium picolinate, can be useful in the present formulations by aiding in glycemic control. After eating, the human body secretes the hormone insulin, which functions to transports glucose to the body's cells to provide energy. Bioavailable chromium sources, particularly chromium picolinate, can stimulate the activity of insulin, which, in principle, aids in the breakdown of glucose and fat. Without being bound by theory or mechanism, it has been reported that bioavailable chromium sources can improve insulin efficiency by increasing the sensitivity of insulin receptors (see Krzanowski, Journal of the Florida Medical Association, 83:1996, pp. 29-31). Further, it has been reported that chromium picolinate can be effective in improving glycemic control and normalizing lipid levels in patients having Type 2 diabetes (see Broadhurst, et al., Diabetes Technol. Ther., 8:2006, pp. 677-687). Improvements in glycemic control can be determined by one or more measures including, for example, fasting glucose levels, postprandial glucose levels, fasting insulin levels, postprandial insulin levels, glycated hemoglobin levels and/or insulin sensitivity.

The present lip cosmetic formulations can contain a bioavailable chromium source, particularly chromium picolinate, in quantities that can be suitable for achieving the foregoing benefits of this substance. That is, the quantity of the bioavailable chromium source can be sufficient to improve glycemic control and lipid levels in a user. In some embodiments, the present formulations can contain between about 0.05% and about 3.0% of the bioavailable chromium source by weight of the formulation, including all values and subranges therebetween. In some embodiments, the present formulations can contain between about 0.05% and about 1.5% of the bioavailable chromium source by weight of the formulation. In other embodiments, the present formulations can contain between about 1.5% and about 3.0% of the bioavailable chromium source by weight of the formulation. In still other embodiments, the present formulations can contain between about 1.5% and about 2.0% of the bioavailable chromium source by weight of the formulation. In still other embodiments, the present formulations can contain between about 0.05% and about 0.5% of the bioavailable chromium source by weight of the formulation. In some embodiments, the present formulations can contain about 0.5% of the bioavailable chromium source by weight of the formulation. In some embodiments, the present formulations can contain up to about 0.5% by weight of the bioavailable chromium source by weight of the formulation.

In some embodiments, the present lip cosmetic formulations can further contain L-carnitine. L-carnitine is an amino acid that is synthesized endogenously in the liver from the natural amino acid precursors lysine and methionine. In the human body, the highest concentrations of this water-soluble amino acid are found in the adrenal glands, the skeletal muscles and the cardiac muscles, with lesser concentrations being found in the kidneys and brain. Without being bound by theory or mechanism, it is believed that L-carnitine can function as a biocatalyst to carry long and medium chain fatty acids across cell walls and into cellular mitochondria, where fatty acids are metabolized for energy production. It is believed that the foregoing distribution of L-carnitine reflects the utilization of fatty acids by these tissues as an energy source. Remaining unbound by theory or mechanism, it is believed that low levels of L-carnitine can result in poor metabolism of fatty acids and buildup within and around cells, which eventually lead to elevated fat and triglyceride levels. L-carnitine can be used in this regard to improve fat metabolism and reduce blood triglyceride levels [see Bremer, et al., J. Physiology Review, 63:1983, pp. 1420-1480; Opie, Am. Heart Journal, 3:1974, pp. 375-377 and Stryer, “Biochemistry,” 3rd ed. (1988, W.H. Freeman and Company, New York)]. Thus, it is believed that L-carnitine can provide weight management effects in the present lip cosmetic formulations. In addition to the foregoing benefits, it is believed that sufficient L-carnitine levels can lead to improved muscle efficiency.

The lip cosmetic formulations of the present disclosure can contain L-carnitine in quantities that can be suitable for achieving the foregoing benefits of this substance. That is, the quantity of L-carnitine can be sufficient to improve fat metabolism and/or reduce blood triglyceride levels in an embodiment. In some embodiments, the present formulations can contain between about 0.05% and about 3.0% L-carnitine by weight of the formulation, including all values and subranges therebetween. In some embodiments, the present formulations can contain between about 0.5% and about 3.0% L-carnitine by weight of the formulation. In some embodiments, the present formulations can contain between about 0.05% and about 1.5% L-carnitine by weight of the formulation. In other embodiments, the present formulations can contain between about 1.5% and about 3.0% L-carnitine by weight of the formulation. In still other embodiments, the present formulations can contain between about 1.5% and about 2.0% L-carnitine by weight of the formulation. In some embodiments, the present formulations can contain about 2.0% L-carnitine by weight of the formulation, or about 1.5% L-carnitine by weight of the formulation, or about 1.0% L-carnitine by weight of the formulation, or about 0.5% L-carnitine by weight of the formulation.

In some embodiments, the lip cosmetic formulations described herein can further include a sunscreen. In some embodiments, the sunscreen can provide a sun protection factor (SPF) of at least about 30. In other embodiments, the sunscreen can provide a SPF of at least about 50.

In general, the sunscreen can contain at least one active ingredient that is suitable for absorbing or reflecting ultraviolet solar radiation, such that the ultraviolet radiation is less likely to result in sunburn of the skin of a wearer. Active ingredients that can be suitable for achieving such effects can include both inorganic and organic compounds. Illustrative but non-limiting active ingredients of a sunscreen that can be suitable for use in the present embodiments can include, for example, ethylhexyl methoxycinnamate and other cinnamic acid derivatives, benzophenones-3 and other benzophenones, octocrylene and other acrylic acid derivatives, ethylhexyl salicylate and other salicylic acid derivatives, octinoxate and octisalate. In various embodiments, any combination of the foregoing sunscreen active ingredients can be used in the present lip cosmetic formulations. In some embodiments, the sunscreen can include octinoxate, octisalate, octocrylene and/or oxybenzone. In various embodiments, each active ingredient of the sunscreen can be present at a concentration ranging between about 1% and about 10% of the lip cosmetic formulation by weight, including all values and subranges therebetween. In other various embodiments, each active ingredient of the sunscreen can be present at a concentration ranging between about 2% and about 8% of the lip cosmetic formulation by weight.

In various embodiments, any number of other components such as, for example, emulsifiers, moisturizers, emollients, flavorants, colorants, menthol, and any combination thereof can be included in the present lip cosmetic formulations. Illustrative examples of each are described hereinafter.

In some embodiments, the lip cosmetic formulations can be formulated in an emollient base to help protect the lips. Illustrative but non-limiting emollient bases can include, for example, waxes and oils (e.g., soybean oils, safflower oils, beeswax, paraffin, coconut oils, and the like). Other suitable emollients can be envisioned by one having ordinary skill in the art. In some embodiments, the emollient base can form between about 50% and about 95% of the lip cosmetic formulation by weight, including all values and subranges therebetween.

In some embodiments, the lip cosmetic formulations can include emulsifiers that aid in mixing with oils. Illustrative but non-limiting emulsifiers can include, for example, polysorbate 60 and lecithin. Other suitable emulsifiers can be envisioned by one having ordinary skill in the art. In some embodiments, the emulsifiers can form between about 0.05% and about 2.0% of the lip cosmetic formulation by weight, including all values and subranges therebetween.

In some embodiments, the lip cosmetic formulations can include moisturizers. Illustrative but non-limiting moisturizers can include, for example, shea butter, coconut oil and aloe barbadensis leaf juice (aloe vera). Other suitable moisturizers can be envisioned by one having ordinary skill in the art. In some embodiments, the moisturizers can form between about 0.05% and about 4.0% of the lip cosmetic formulation by weight, including all values and subranges therebetween.

In some embodiments, the lip cosmetic formulations can include flavorants and/or colorants. Colorants can alter the aesthetic appearance of the lip cosmetic formulations. Flavorants can alter or mask the taste or odor of any of any components within the lip cosmetic formulations and improve the wearer's experience. Such colorants and flavorants can be natural or artificial compounds. Illustrative colorants can include, for example, Red 6 Lake (Cl 15850), Blue 1 Lake (Cl 42090), Violet 2 (Cl 60725) and Yellow 5 Lake (Cl 19140). In some embodiments, the flavorants and/or colorants can form between about 0.05% and about 1.5% of the lip cosmetic formulation by weight, including all values and subranges therebetween.

In some embodiments, the lip cosmetic formulations can include menthol. Benefits of including menthol in the present formulations can include, for example, improved vasodilatory action and a cooling sensation when applied to the lips. In some embodiments, menthol can form between about 0.05% and about 1.0% of the lip cosmetic formulation by weight, including all values and subranges therebetween.

In some embodiments, the lip cosmetic formulations can include antimicrobial agents such as, for example, parabens (e.g., methylparaben, ethylparaben and propylparaben). In some embodiments, the antimicrobial agents can form between about 0.05% and about 0.5% of the lip cosmetic formulation by weight, including all values and subranges therebetween.

In some embodiments, lip cosmetic formulations described herein can include caffeine, extract of Hoodia gordinii and extract of green tea, where a concentration of the caffeine, the extract of Hoodia gordinii and the extract of green tea can each range between about 1.5% and about 2.0% by weight of the lip cosmetic formulation.

In some embodiments, lip cosmetic formulations described herein can include caffeine, extract of Hoodia gordinii, extract of green tea and chromium picolinate, where a concentration of the caffeine, the extract of Hoodia gordinii and the extract of green tea can each range between about 1.5% and about 2.0% by weight of the lip cosmetic formulation and a concentration of the chromium picolinate can range between about 0.05% and about 0.5% by weight of the lip cosmetic formulation.

In one embodiment, a lip cosmetic formulation of the present disclosure can be formulated as set forth in Table 1. This lip cosmetic formulation can provide a SPF of about 15.

TABLE 1

| Ingredient | Concentration (% by wt.) |
|---|---|
| ethylhexyl methoxycinnamate | 7.00 |
| benzophenone-3 | 4.50 |
| soybean wax | 34.00 |
| safflower seed oil | 33.65 |
| beeswax | 5.75 |
| paraffin | 5.75 |
| coconut oil | 3.25 |
| polysorbate 60 | 1.50 |
| shea butter | 0.75 |
| caffeine | 0.75 |
| green tea extract | 0.75 |
| Hoodia gordonii extract | 0.75 |
| chromium picolinate | 0.50 |
| lecithin | 0.50 |
| menthol | 0.25 |
| propylparaben | 0.10 |
| flavorant | 0.25 |

In one embodiment, a lip cosmetic formulation of the present disclosure can be formulated as set forth in Table 2. This lip cosmetic formulation can provide a SPF of about 30.

TABLE 2

| Ingredient | Concentration (% by wt.) |
|---|---|
| ethylhexyl methoxycinnamate | 7.50 |
| benzophenone-3 | 6.00 |
| octocrylene | 6.00 |
| ethylhexyl salicylate | 3.00 |
| soybean wax | 27.00 |
| safflower seed oil | 26.85 |
| beeswax | 5.75 |
| paraffin | 5.75 |
| coconut oil | 3.25 |
| polysorbate 60 | 1.50 |
| caffeine | 1.25 |
| green tea extract | 1.25 |
| aloe barbadensis leaf juice | 0.75 |
| shea butter | 0.75 |
| Hoodia gordonii extract | 0.75 |
| chromium picolinate | 0.50 |
| L-carnitine | 0.50 |
| lecithin | 0.50 |
| menthol | 0.25 |
| propylparaben | 0.10 |
| flavorant | 0.25 |
| Yellow 5 | 0.25 |
| Red 7 | 0.15 |
| castor oil | 0.15 |

In one embodiment, a lip cosmetic formulation of the present disclosure can be formulated as set forth in Table 3. This lip cosmetic formulation can provide a SPF of about 30.

TABLE 3

| Ingredient | Concentration (% by wt.) |
|---|---|
| ethylhexyl methoxycinnamate | 7.50 |
| benzophenone-3 | 6.00 |
| octocrylene | 6.00 |
| ethylhexyl salicylate | 3.00 |
| soybean wax | 27.75 |
| safflower seed oil | 27.10 |
| beeswax | 5.75 |
| paraffin | 5.75 |
| coconut oil | 3.25 |
| polysorbate 60 | 1.50 |
| caffeine | 1.25 |
| green tea extract | 1.25 |
| aloe barbadensis leaf juice | 0.75 |
| shea butter | 0.75 |
| Hoodia gordonii extract | 0.75 |
| lecithin | 0.50 |
| menthol | 0.25 |
| propylparaben | 0.10 |
| flavorant | 0.25 |
| Yellow 5 | 0.25 |
| Blue 1 | 0.15 |
| castor oil | 0.15 |

In one embodiment, a lip cosmetic formulation of the present disclosure can be formulated, where the Hoodia Gordonii extract, the caffeine and the green tea extract are in a safflower seed oil base. In one embodiment, such a lip cosmetic formulation can be formulated as set forth in Table 4.

TABLE 4

| Ingredient | Concentration (% by wt.) |
|---|---|
| soybean oil | 33.65 |
| beeswax | 5.75 |
| safflower seed oil/Hoodia | 2.00 |
| gordonii extract | |
| safflower seed oil/caffeine | 2.00 |
| safflower seed oil/green tea | 2.00 |
| extract | |
| safflower seed oil | 33.65 |
| carnauba wax | 1.25 |
| paraffin | 5.75 |
| flavorant | 2.00 |
| coconut oil | 3.25 |
| coconut oil/Stevia rebaudiana | 3.25 |
| leaf and stem extract | |
| shea butter | 0.75 |
| lecithin | 0.50 |
| phenoxyethanol | 0.20 |
| castor seed oil/iron oxide | 2.00 |
| (Cl 77499) | |
| Red 6 Lake (Cl 15850) | 2.00 |

In some embodiments, a lip cosmetic formulation, including any of those set forth above, can be formulated such that it has a SPF of about 30. In some embodiments, an SPF of about 30 can be imparted to the lip cosmetic formulations using the following components by weight: about 7.50% octinoxate, about 5.00% octisalate, about 7.50% octocrylene and about 5.00% oxybenzone. In some embodiments, a lip cosmetic formulation with an SPF of about 30 can have the concentration of Hoodia gordonii extract, caffeine and green tea extract held constant relative to a lip cosmetic formulation lacking a sunscreen, with the weight percentages of other components making up the balance of the lip cosmetic formulation being adjusted downward. For example, the lip cosmetic formulation set forth in Table 4 can have components added to impart a SPF of about 30, while the concentrations of all components except Hoodia gordonii extract, caffeine and green tea extract are adjusted downward.

It is believed that the foregoing combinations of active substances can result in weight loss and weight management when included in a lip cosmetic formulation and applied regularly by a user. Although any one of the active substances can potentially provide some beneficial effects to a user, it is believed that the combined use of the active substances in a single formulation can beneficially promote more effective weight loss and weight management through multiple routes. According to the present embodiments, routes through which weight loss and weight management can occur include, for example, appetite suppression, fat burning, lean muscle building and improving the user's energy levels. In some embodiments, it is believed that the various active components of the present formulations can interact synergistically with one another such that greater weight loss and weight management properties can be realized than through use of less than all of the active ingredients.

In various embodiments, the present lip cosmetic formulations can be prepared by techniques which are known to one having ordinary skill in the art. In some embodiments, powdered Hoodia gordonii and green tea extracts can be taken up in a carrier oil such as, for example, safflower seed oil, prior to mixing with the remaining components. In other embodiments, the caffeine and Hoodia gordonii and green tea extracts can be provided in liquid form (e.g., in a safflower seed oil base). Optionally, the liquid form of these extracts can be taken up in another carrier oil prior to mixing with the remaining components. In either case, the resultant lip cosmetic formulations can have a smooth, non-grainy texture and feel. In some embodiments, pre-dissolving the extracts in a carrier oil can enhance the texture and feel of the lip cosmetic formulations. In addition, it is believed that pre-dissolution of the extracts in a carrier oil can enhance their bioavailability.

All publications referenced herein are incorporated by reference in their entirety.

It is understood that modifications which do not substantially affect the activity of the various embodiments of this invention are also included within the definition of the invention provided herein. Accordingly, the following Examples are intended to illustrate but not limit the present invention.

### EXAMPLE 1

### Clinical Testing of a Lip Balm Formulation

Double blind placebo-controlled clinical trials were conducted on approximately 120 patients using a lip balm formulated as described herein. Over the course of the twelve-week study, patients regularly using the lip balm formulations lost an average of 7 to 9 more pounds than did the placebo group. Patients in the study group reported satiation with smaller food portions and a reduced propensity to snack.

More specifically, 120 study participants age 18 to 62 who were classified as overweight (BMI 25-30), obese (BMI 30-40) or very obese (BMI 40-50) were randomized into six study groups. Three study groups were given the present lip cosmetic formulation, and three study groups were given a placebo lip cosmetic formulation. The weight of the study participants was monitored on a weekly, bi-weekly or monthly basis for a total of twelve weeks. All study participants were instructed to use the lip cosmetic formulation at least four times per day but no more than eight times per day.

Study participants classified as overweight reported an average weight loss of 7.17 pounds, while the corresponding placebo group lost an average of 1.55 pounds. Study participants classified as obese reported an average weight loss of 8.86 pounds, while the corresponding placebo group lost an average of 1.64 pounds. Study participants classified as very obese reported an average weight loss of 9.43 pounds, while the corresponding placebo group lost an average of 1.43 pounds.

Although the invention has been described with reference to the disclosed embodiments, those of ordinary skill in the art will readily appreciate that these embodiments are only illustrative of the invention. It should be understood that various modifications can be made without departing from the spirit of the invention. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. While compositions and methods are described in terms of “comprising,” “containing,” or “including” various components or steps, the compositions and methods can also “consist essentially of” or “consist of” the various components and operations. All numbers and ranges disclosed above can vary by some amount. Whenever a numerical range with a lower limit and an upper limit is disclosed, any number and any subrange falling within the broader range is specifically disclosed. Also, the terms in the claims have their plain, ordinary meaning unless otherwise explicitly and clearly defined by the patentee. If there is any conflict in the usages of a word or term in this specification and one or more patent or other documents that may be incorporated herein by reference, the definitions that are consistent with this specification should be adopted.

What is claimed is the following:

1. A lip cosmetic formulation comprising:
caffeine;
extract of Hoodia gordonii; and
extract of green tea.

2. The lip cosmetic formulation of claim 1, wherein a concentration of the caffeine, the extract of Hoodia gordonii and the extract of green tea each range between about 0.5% and about 3.0% by weight of the lip cosmetic formulation.

3. The lip cosmetic formulation of claim 2, further comprising:
a bioavailable chromium source.

4. The lip cosmetic formulation of claim 3, wherein the bioavailable chromium source comprises chromium picolinate.

5. The lip cosmetic formulation of claim 4, wherein a concentration of the chromium picolinate ranges between about 0.05% and about 3.0% by weight of the lip cosmetic formulation.

6. The lip cosmetic formulation of claim 3, further comprising:
a sunscreen.

7. The lip cosmetic formulation of claim 6, wherein the sunscreen provides a sun protection factor of at least about 30.

8. The lip cosmetic formulation of claim 3, further comprising:
L-carnitine.

9. The lip cosmetic formulation of claim 8, wherein a concentration of the L-carnitine ranges between about 0.5% and about 3.0% by weight of the lip cosmetic formulation.

10. The lip cosmetic formulation of claim 8, further comprising:
a sunscreen.

11. The lip cosmetic formulation of claim 10, wherein the sunscreen provides a sun protection factor of at least about 30.

12. The lip cosmetic formulation of claim 2, further comprising:
a sunscreen.

13. The lip cosmetic formulation of claim 12, wherein the sunscreen provides a sun protection factor of at least about 30.

14. The lip cosmetic formulation of claim 1, further comprising:
at least one component selected from the group consisting of an emulsifier, a moisturizer, an emollient, a flavorant, a colorant, menthol, and any combination thereof.

15. A lip cosmetic formulation comprising:
caffeine;
extract of Hoodia gordonii; and
extract of green tea;
wherein a concentration of the caffeine, the extract of Hoodia gordoni and the extract of green tea each range between about 1.5% and about 2.0% by weight of the lip cosmetic formulation.

16. The lip cosmetic formulation of claim 15, further comprising:
a sunscreen.

17. The lip cosmetic formulation of claim 16, wherein the sunscreen provides a sun protection factor of at least about 30.

18. A lip cosmetic formulation comprising:
caffeine;
extract of Hoodia gordonii;
extract of green tea; and
chromium picolinate;
wherein a concentration of the caffeine, the extract of Hoodia gordoni and the extract of green tea each range between about 1.5% and about 2.0% by weight of the lip cosmetic formulation and a concentration of the chromium picolinate ranges between about 0.05% and about 0.5% by weight of the lip cosmetic formulation.

19. The lip cosmetic formulation of claim 18, further comprising:
a sunscreen.

20. The lip cosmetic formulation of claim 19, wherein the sunscreen provides a sun protection factor of at least about 30.

21. The lip cosmetic formulation of claim 18, further comprising:
L-carnitine.

22. The lip cosmetic formulation of claim 21, wherein a concentration of the L-carnitine ranges between about 1.5% and about 2.0% by weight of the lip cosmetic formulation.
